(19) United States
(12) Patent Application Publication
(10) Pub. No.: US 2024/0101522 A1
(43) Pub. Date: Mar. 28, 2024

(54) NOVEL METHOD FOR PRODUCING BENZOXAZINE COMPOUND

(71) Applicant: HONSHU CHEMICAL INDUSTRY CO., LTD., Chuo-ku, Tokyo (JP)

(72) Inventor: Yoshimi UTAKA, Wakayama-shi (JP)

(21) Appl. No.: 18/262,136

(22) PCT Filed: Jan. 24, 2022
(86) PCT No.: PCT/JP2022/002324

§ 371 Date: Jul. 19, 2023

(30) Foreign Application Priority Data
Jan. 29, 2021 (JP) 2021-013398
Aug. 24, 2021 (JP) 2021-136251

(51) Int. Cl. C07D 265/16

(52) U.S. Cl. CPC C07D 265/16

(57) ABSTRACT

A method for producing a benzoxazine compound represented by general formula (3), includes performing a reaction by mixing a mixture containing a bisphenol compound represented by general formula (1) and a formaldehyde with an amine compound represented by general formula (2):

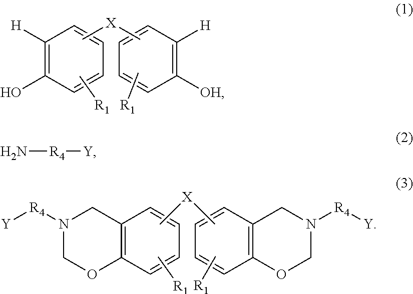

### TECHNICAL FIELD

The present invention relates to a novel method for producing a benzoxazine compound. The invention particularly relates to a novel method for producing a benzoxazine compound having benzoxazine rings at both ends of a linking group and further having a hydroxy group or a thiol group.

### BACKGROUND ART

Benzoxazine compounds, which are compounds synthesized by reacting a phenol, an amine, and formaldehyde, are known as thermosetting resin raw materials that, when heated, undergo ring-opening polymerization of a benzoxazine ring to cure without producing any volatile by-products, and are used as raw materials of a molded body usable as a material for an insulating substrate, a liquid crystal alignment agent, a resin composition for semiconductor sealing, and the like. In these applications, heat resistance excellent in stability and reliability at high temperatures is required.

On the other hand, benzoxazine compounds typically have relatively high curing temperatures, and to achieve lower polymerization temperatures, catalysts, polymerization accelerators, and in addition highly reactive benzoxazine compounds have recently been developed. Among the highly reactive benzoxazine compounds, a benzoxazine composition that can be cured in an environmentally friendly process at relatively low temperatures in short time periods and contains a hydroxy group or a nitrogen-containing heterocycle has been reported (PTL 1).

Examples of known methods for synthesizing a benzoxazine compound containing a hydroxy group include a synthesis method in which bisphenol A, an alcohol amine monomer, and paraformaldehyde, which are raw materials, are mixed at one time together with a solvent and allowed to react (PTL 2) and a synthesis method in which a mixed solution of paraformaldehyde and ethanolamine is first prepared, and then a solution of bisphenol A is added and allowed to react (NPL 1). It has been reported that in these methods, the synthesis is performed at a high temperature of 90° C. or higher, and the product yield is relatively high.

### CITATION LIST

### Patent Literature

PTL 1: Japanese Unexamined Patent Application Publication (Translation of PCT Application) No. 2011-530570

PTL 2: Chinese Patent Application Publication No.

### Non Patent Literature

NPL 1: E. Gilbert et al., J. Polym. Res. 2018, Vol. 25, p. 114.

### SUMMARY OF INVENTION

### Technical Problem

The present inventors have attempted to synthesize a benzoxazine compound having a hydroxy group by the above production methods known in the art, and observed a phenomenon in which the temperature of a mixed solution rapidly increases when raw materials are mixed and smoke generates inside a reactor to cause an increase in internal pressure. In consideration of implementation of industrial production as well as the operation at a laboratory level, conditions such as a rapid temperature increase and smoke generation at the time of raw material loading should be avoided as much as possible because such conditions have disadvantages of causing spouting of components in a reactor due to a rapid increase in internal pressure of the reactor and necessitating energy and time for thermal control.

One widely accepted mechanism of production of a benzoxazine compound is a production mechanism including a first step in which an amine is reacted with a formaldehyde to form an intermediate compound having a hexahydrotriazine structure and a second step in which the intermediate compound is reacted with a phenol and a formaldehyde to form a compound having a benzoxazine structure (e.g., paragraph 0005 of Japanese Unexamined Patent Application Publication (Translation of PCT Application) No. 2017-537182).

The present inventors have presumed that reaction heat that generates when the amine is reacted with the formaldehyde to form an intermediate compound having a hexahydrotriazine structure as described above is the cause of the rapid temperature increase and smoke generation at the time of raw material loading.

It is an object of the present invention to provide a method for producing a target benzoxazine compound with higher safety and high efficiency without causing a rapid temperature increase or smoke generation at the time of raw material loading before the formation of the benzoxazine compound.

### Solution to Problem

To achieve the above object, the present inventors have conducted intensive studies and found that the above problem can be solved and the target benzoxazine compound can be synthesized by changing the method of mixing raw materials, particularly, by mixing a bisphenol compound and a formaldehyde and then mixing an amine, thereby completing the present invention.

It has been confirmed that heat generation is not observed at the stage of mixing the bisphenol compound and the formaldehyde, and upon mixing the amine thereafter, the reaction to obtain the target benzoxazine compound proceeds.

The present invention is as follows.
1. A method for producing a benzoxazine compound represented by general formula (3), the method including performing a reaction by mixing a mixture containing a bisphenol compound represented by general formula (1) and a formaldehyde with an amine compound represented by general formula (2).

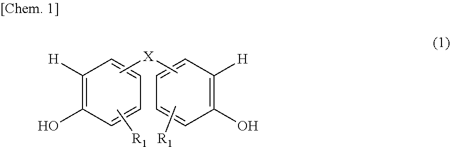

(In the formula, R₁ represents a hydrogen atom or an alkyl group having 1 to 6 carbon atoms, and X represents a single bond, an oxygen atom, a sulfur atom, a sulfonyl group, a carbonyl group, or a divalent group represented by general formula (1a) or (1b).)

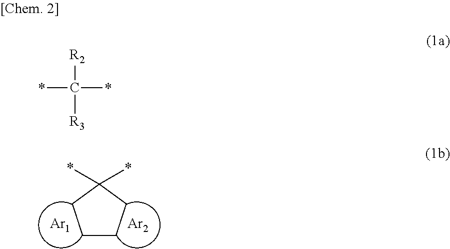

(In general formulae (1a) and (1b), R₂ and R₃ each independently represent hydrogen, an alkyl group having 1 to 10 carbon atoms, an alkyl halide group having 1 to 10 carbon atoms, or an aryl group having 6 to 12 carbon atoms, R₂ and R₃ are optionally bonded to each other to together form a cycloalkylidene group having 5 to 20 carbon atoms, Ar₁ and Ar₂ each independently represent an aryl group having 6 to 12 carbon atoms, and * represents a bonding position.)

[Chem. 3]

H₂N—R₄—Y (2)

(In the formula, R₄ represents a divalent group having 1 to 10 carbon atoms, and Y represents a hydroxy group or a thiol group.)

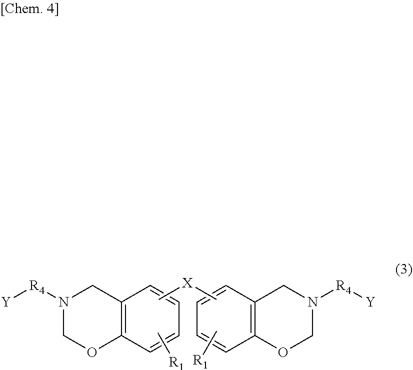

(In the formula, R₁ and X represent the same groups as in general formula (1), and R₄ and Y represent the same groups as in general formula (2).)

### Advantageous Effects of Invention

According to the method for producing a benzoxazine compound according to the present invention, the target benzoxazine compound can be easily obtained with higher safety and high efficiency in industrial production without causing a rapid temperature increase or smoke generation at the time of raw material loading before the formation of the benzoxazine compound, which is very useful.

### DESCRIPTION OF EMBODIMENTS

### Method for Producing Benzoxazine Compound According to Present Invention

A method for producing a benzoxazine compound according to the present invention includes performing a reaction by mixing a mixture containing a bisphenol compound represented by general formula (1) and a formaldehyde with an amine compound represented by general formula (2), and a benzoxazine compound represented by general formula (3) is the target compound of the production method.

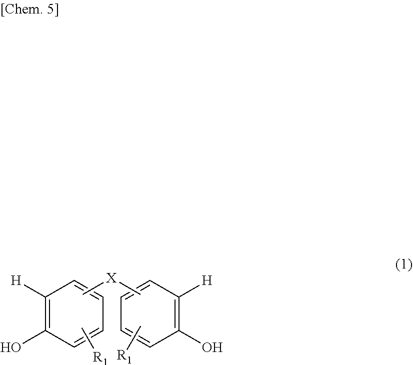

(In the formula, R₁ represents a hydrogen atom or an alkyl group having 1 to 6 carbon atoms, and X represents a single bond, an oxygen atom, a sulfur atom, a sulfonyl group, a carbonyl group, or a divalent group represented by general formula (1a) or (1b).)

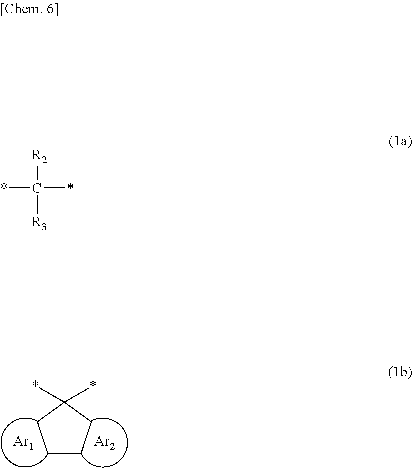

(In general formulae (1a) and (1b), R₂ and R₃ each independently represent hydrogen, an alkyl group having 1 to 10 carbon atoms, an alkyl halide group having 1 to 10 carbon atoms, or an aryl group having 6 to 12 carbon atoms, R₂ and R₃ are optionally bonded to each other to together form a cycloalkylidene group having 5 to 20 carbon atoms, Ari and Are each independently represent an aryl group having 6 to 12 carbon atoms, and * represents a bonding position.)

[Chem. 7]

H₂N—R₄—Y (2)

(In the formula, R₄ represents a divalent group having 1 to 10 carbon atoms, and Y represents a hydroxy group or a thiol group.)

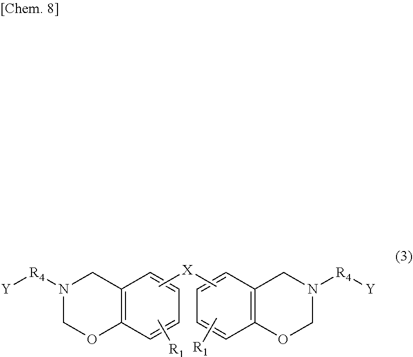

(In the formula, R₁ and X represent the same groups as in general formula (1), and R₄ and Y represent the same groups as in general formula (2).)

R₁ in general formulae (1) and (3) is preferably a hydrogen atom or an alkyl group having 1 to 4 carbon atoms, more preferably a hydrogen atom or an alkyl group having 1 carbon atom (methyl group), particularly preferably a hydrogen atom. When R₁ is not a hydrogen atom, the bonding position thereof is preferably the ortho position to each hydroxy group in general formula (1), and is preferably the ortho position on the benzene ring relative to the oxygen atom of each benzoxazine ring in general formula (3).

When X in general formulae (1) and (3) is represented by general formula (1a), R₂ and R₃, independently of each other, are more preferably hydrogen, an alkyl group having 1 to 6 carbon atoms, an alkyl halide group having 1 to 6 carbon atoms, or an aryl group having 6 to 12 carbon atoms, still more preferably hydrogen, an alkyl group having 1 to 4 carbon atoms, a trifluoromethyl group, or an aryl group having 6 to 8 carbon atoms, particularly preferably hydrogen, an alkyl group having 1 to 4 carbon atoms, or a phenyl group.

R₂ and R₃ may be bonded to each other to together form a cycloalkylidene group having 5 to 20 carbon atoms. The cycloalkylidene group having 5 to 20 carbon atoms may include a branched-chain alkyl group. The cycloalkylidene group preferably has 5 to 15 carbon atoms, more preferably has 6 to 12 carbon atoms, and particularly preferably has 6 to 9 carbon atoms.

Specific examples of the cycloalkylidene group include a cyclopentylidene group (5 carbon atoms), a cyclohexylidene group (6 carbon atoms), a 3-methylcyclohexylidene group (7 carbon atoms), a 4-methylcyclohexylidene group (7 carbon atoms), a 3,3,5-trimethylcyclohexylidene group (9 carbon atoms), a cycloheptylidene group (7 carbon atoms), a bicyclo[2.2.1]heptane-2,2-diyl group (7 carbon atoms), a 1,7,7-trimethylbicyclo[2.2.1]heptane-2,2-diyl group (10 carbon atoms), a 4,7,7-trimethylbicyclo[2.2.1]heptane-2,2-diyl group (10 carbon atoms), a tricyclo[5.2.1.0 2,6 ]decane-8,8-diyl group (10 carbon atoms), a 2,2-adamantylidene group (10 carbon atoms), and a cyclododecanylidene group (12 carbon atoms). Preferred are a cyclohexylidene group (6 carbon atoms), a 3-methylcyclohexylidene group (7 carbon atoms), a 4-methylcyclohexylidene group (7 carbon atoms), a 3,3,5-trimethylcyclohexylidene group (9 carbon atoms), and a cyclododecanylidene group (12 carbon atoms), more preferred are a cyclohexylidene group (6 carbon atoms), a 3,3,5-trimethylcyclohexylidene group (9 carbon atoms), and a cyclododecanylidene group (12 carbon atoms), and particularly preferred are a cyclohexylidene group (6 carbon atoms) and a 3,3,5-trimethylcyclohexylidene group (9 carbon atoms).

When X in general formulae (1) and (3) is represented by general formula (1b), Ar₁ and Ar₂ are preferably each independently a benzene ring or a naphthalene ring, and Ar₁ and Ar₂ are more preferably each a benzene ring. For example, when Ar₁ and Ar₂ are each a benzene ring, the group represented by general formula (1b) is a fluorenylidene group.

The position of bonding of X in general formula (3) to the two benzoxazine rings is preferably the ortho or para position on the benzene ring relative to the oxygen atom of each benzoxazine ring, and the position of bonding of X on the benzene ring in general formula (1), which is the material of general formula (3), is also preferably the ortho or para position relative to each hydroxy group.

R₄ in general formulae (2) and (3) is a divalent group having 1 to 10 carbon atoms, and specific examples include linear or branched alkylene groups having 1 to 10 carbon atoms or alkylene groups including a cyclic alkane, such as a methylene group, an ethylene group, a propane-1,2-diyl group, a propane-1,3-diyl group, a butane-1,4-diyl group, a pentane-1,5-diyl group, a hexane-1,6-diyl group, a cyclohexane-1,3-diyl group, and a cyclohexane-1,4-diyl group; alkylidene groups having 1 to 10 carbon atoms such as an ethylidene group, a propylidene group, an isopropylidene group, a butylidene group, a cyclopentylidene group, and a cyclohexylidene group; a phenylene group; and divalent groups containing a benzene ring and having 1 to 10 carbon atoms, such as groups represented by the following formulae.

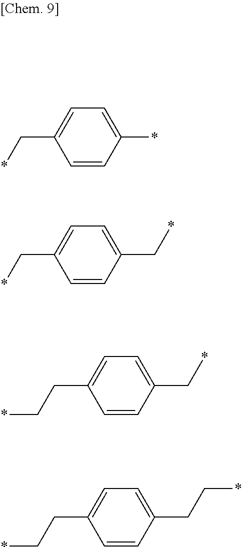 [SMILES: *Cc1ccc(*)cc1 ; *Cc1ccc(C*)cc1 ; *CCc1ccc(C*)cc1 ; *CCc1ccc(CC*)cc1]

(In the formulae, * represents a bonding position.)

Of these, R₄ is preferably a linear or branched alkylene group having 1 to 10 carbon atoms, an alkylene group including a cyclic alkane, or an alkylidene group having 1 to 10 carbon atoms, more preferably a linear or branched alkylene group having 1 to 10 carbon atoms or an alkylene group including a cyclic alkane, still more preferably a linear or branched alkylene group having 1 to 6 carbon atoms or an alkylene group including a cyclic alkane, particularly preferably a linear or branched alkylene group having 1 to 4 carbon atoms.

Specific examples of the benzoxazine compound represented by general formula (3), which is the target compound of the production method according to the present invention, include compounds (p-1) to (p-6) having the following chemical structures.

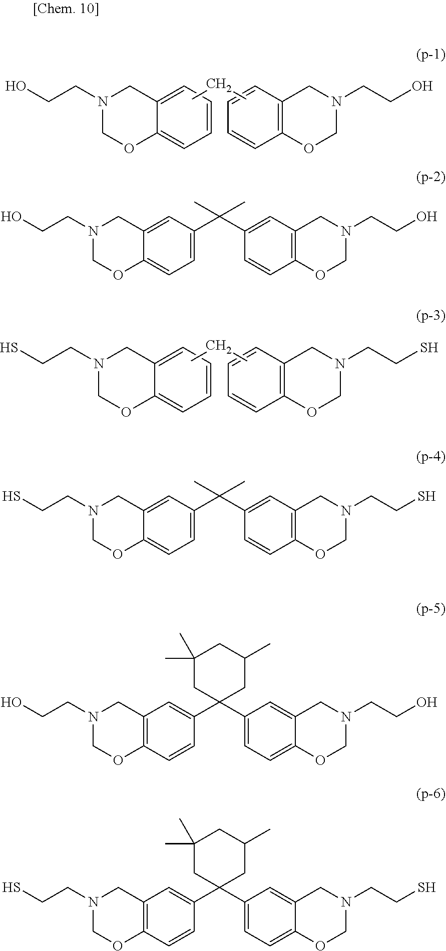

### <Starting Material: Bisphenol Compound Represented by General Formula (1)>

Specific examples of the bisphenol compound represented by general formula (1), which is a starting material in the method for producing a benzoxazine compound according to the present invention, include bisphenol F (bis(2-hydroxyphenyl)methane, 2-hydroxyphenyl-4-hydroxyphenylmethane, bis(4-hydroxyphenyl)methane), bisphenol E (1,1-bis(4-hydroxyphenyl)ethane), bisphenol A (2,2-bis(4-hydroxyphenyl)propane), bisphenol C (2,2-bis(4-hydroxy-3-methylphenyl)propane), 2,2-bis(4-hydroxyphenyl)-4-methylpentane, 4,4′-dihydroxybiphenyl, 4,4′-dihydroxy-3,3′-dimethylbiphenyl, bis(4-hydroxyphenyl) ether, 4,4′-dihydroxybenzophenone, bis(4-hydroxyphenyl)sulfone, bis(4-hydroxyphenyl) sulfide, 1,1-bis(4-hydroxyphenyl)-1-phenylethane, 1,1-bis(4-hydroxyphenyl)-1-naphthylethane, 2,2-bis(4-hydroxyphenyl)hexafluoropropane, bisphenol M (1,3-bis(2-(4-hydroxyphenyl)-2-propyl)benzene), bisphenol Z (1,1-bis(4-hydroxyphenyl)cyclohexane), bisphenol TMC (1,1-bis(4-hydroxyphenyl)-3,3,5-trimethylcyclohexane), 1,1-bis(4-hydroxyphenyl)cyclododecane, 2,2-bis(4-hydroxyphenyl)adamantane, and 9,9-bis(4-hydroxy-3-methylphenyl)fluorene.

### <Starting Material: Formaldehyde>

Specific examples of the formaldehyde, which is a starting material in the novel method for producing a benzoxazine compound according to the present invention, include an aqueous formaldehyde solution, 1,3,5-trioxane, and paraformaldehyde.

### <Starting Material: Amine Compound Represented by General Formula (2)>

Specific examples of the amine compound represented by general formula (2), which is a starting material in the method for producing a benzoxazine compound according to the present invention, include the following compounds.

Specific examples of cases where “Y” in general formula (2) is a hydroxy group include methanolamine, 2-aminoethanol, 1-amino-2-propanol, 2-amino-1-methylethanol, 2-amino-2-methylethanol, 3-amino-1-propanol, 4-amino-1-butanol, 2-amino-1-butanol, 4-amino-2-butanol, 5-amino-1-pentanol, 6-amino-1-hexanol, 7-amino-1-heptanol, valinol, 2-aminophenol, 3-aminophenol, 4-aminophenol, and 4-aminobenzyl alcohol. Of these, 2-aminoethanol, 2-amino-1-methylethanol, 2-amino-2-methylethanol, 3-amino-1-propanol, 2-aminophenol, 3-aminophenol, and 4-aminophenol are preferred, 2-aminoethanol, 2-aminophenol, 3-aminophenol, and 4-aminophenol are more preferred, and 2-aminoethanol is particularly preferred.

Specific examples of cases where “Y” in general formula (2) is a thiol group include 2-aminoethanethiol, 3-amino-1-propanethiol, 2-amino-l-methylethanethiol, 2-amino-2-methylethanethiol, 5-amino-1-pentanethiol, 6-amino-1-hexanethiol, 2-aminothiophenol, 3-aminothiophenol, 4-aminothiophenol, and 4-aminobenzyl mercaptan. Of these, 2-aminoethanethiol, 3-amino-1-propanethiol, 2-aminothiophenol, 3-aminothiophenol, and 4-aminothiophenol are preferred, 2-aminoethanethiol, 2-aminothiophenol, 3-aminothiophenol, and 4-aminothiophenol are more preferred, and 2-aminoethanethiol is particularly preferred.

### <Amount of Starting Material Used>

In the production method according to the present invention, the amount of the formaldehyde used is preferably in the range of 4.0 to 20.0 mol, more preferably in the range of 4.0 to 16.0 mol, still more preferably in the range of 4.0 to 12.0 mol, relative to 1 mol of the bisphenol compound represented by general formula (1).

In the production method according to the present invention, the amount of the amine compound represented by general formula (2) used is preferably in the range of 2.0 to 10.0 mol, more preferably in the range of 2.0 to 8.0 mol, still more preferably in the range of 2.0 to 6.0 mol, relative to 1 mol of the bisphenol compound represented by general formula (1).

### <Catalyst>

In the production method according to the present invention, a catalyst for accelerating the reaction is not particularly necessary, but an acid catalyst or a base catalyst can be used as needed. In this case, examples of acid catalysts that can be used include, but are not limited to, concentrated hydrochloric acid, hydrochloric acid gas, trifluoroacetic acid, methanesulfonic acid, p-toluenesulfonic acid, benzoic acid, and mixtures thereof, and examples of base catalysts that can be used include, but are not limited to, sodium hydroxide, sodium carbonate, triethylamine, triethanolamine, and mixtures thereof.

### <Solvent>

In the production method according to the present invention, the reaction is typically performed in the presence of a solvent. The solvent is not particularly limited as long as it does not inhibit the reaction, and preferred examples include toluene, xylene, ethyl acetate, butyl acetate, chloroform, dichloromethane, tetrahydrofuran, and dioxane. These solvents can be used alone or in combination. The amount of solvent used is not particularly limited as long as the reaction is not hindered, and is typically in the range of 0.5 to 5 times, preferably in the range of 1 to 3 times the amount of the bisphenol compound represented by general formula (1) on a weight basis.

### <Reaction Temperature>

In the production method according to the present invention, the reaction can be performed typically at a temperature in the range of 10° C. to 150° C.

From the viewpoint of reaction selectivity of the benzoxazine compound represented by general formula (3), which is the target compound, suppression of formation of by-product high-molecular-weight components, and suppression of viscosity increase or solidification of a solution after the reaction, the reaction temperature is preferably in the range of 10° C. to 80° C., more preferably in the range of 20° C. to 70° C., still more preferably in the range of 20° C. to 60° C., particularly preferably in the range of 20° C. to 40° C.

### <Reaction Pressure>

In the production method according to the present invention, the reaction may be performed under normal pressure conditions, or may be performed under increased pressure or reduced pressure.

In another embodiment, a process of removing water derived from the raw materials or water generated during the reaction out of the system may be included. The process of removing water generated from a reaction solution is not particularly limited and can be performed by distilling the generated water azeotropically with the solvent system in the reaction solution. The generated water can be removed out of the reaction system by using, for example, an isobaric dropping funnel equipped with a cock, a Dimroth condenser, or a Dean-Stark apparatus.

The mixture containing a bisphenol compound represented by general formula (1) and a formaldehyde can be obtained by mixing the bisphenol compound represented by general formula (1) and the formaldehyde. The method of the mixing is not particularly limited, and, for example, the formaldehyde may be added into a reactor containing the bisphenol compound represented by general formula (1), and vice versa. This mixture may contain the solvent and the catalyst described above. The method of mixing them is not particularly limited, but the catalyst is preferably mixed before the amine compound represented by general formula (2) is mixed.

In the production method according to the present invention, this mixture is mixed with the amine compound represented by general formula (2) to perform the reaction. The method of mixing the amine compound is not particularly limited, but since the formation of the benzoxazine compound represented by general formula (3) is a reaction accompanied by heat generation, from the viewpoint of reaction selectivity and suppression of formation of by-product high-molecular-weight components, the amine compound is preferably mixed continuously or intermittently so as to prevent a rapid increase in the temperature of the reaction solution.

### <After Completion of Reaction>

From the final reaction mixture obtained by the production method according to the present invention, the benzoxazine compound represented by general formula (3) can be obtained by a known method after completion of the reaction. For example, after the reaction, the remaining raw materials and solvent may be distilled off from the reaction mixture to thereby obtain the target as a residual liquid. Other possible methods include adding the residual liquid to a poor solvent to obtain the target as a precipitate, and adding a solvent to the reaction mixture to cause crystallization and performing filtration to obtain the target as powder or particles. The benzoxazine compound collected by any of these methods can be made into a high-purity product by, for example, standard purification means such as washing with a solvent or water or recrystallization.

### EXAMPLES

The present invention will now be described more specifically with reference to Examples.

### Analysis Method

### 1. Reaction Solution Composition and Purity Analysis (Gel Permeation Chromatography: GPC)

The purity of benzoxazine compounds synthesized by the production method according to the present invention was defined as the area percentage of the benzoxazine compounds determined by this analysis.
Apparatus: HLC-8320/manufactured by Tosoh Corporation
Detector: differential refractometer (RI)

### [Measurement Conditions]

Flow rate: 1 mL/min
Eluate: tetrahydrofuran
Temperature: 40 ° C.
Wavelength: 254 nm
Measurement sample: One gram of a benzoxazine compound-containing composition was 200-fold diluted with tetrahydrofuran.

### <Example 1> (Synthesis of Compound p-1 by Production Method According to Present Invention)

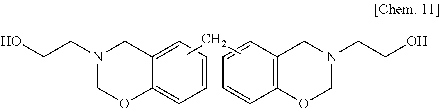

In a 1 L four-necked flask equipped with a thermometer, a stirrer, a condenser, and a dropping funnel, 97 g (0.48 mol) of bisphenol F (binuclear structure content, 90.1 wt %; isomer ratio thereof: bis(2-hydroxyphenyl)methane, 18.8 wt %; 2-hydroxyphenyl-4-hydroxyphenylmethane, 49.3 wt %; and bis(4-hydroxyphenyl)methane, 31.9 wt %; polynuclear structure content, 9.9 wt %), 62 g of 94% paraformaldehyde, and 121 g of toluene were loaded. During the loading, heat generation was not observed, and as a result of analyzing the mixed solution by high-performance liquid chromatography (HPLC), only the raw materials used were detected. After the reactor was purged with nitrogen, the temperature of the mixed solution was adjusted to 70° C. While maintaining the temperature, 60 g of 2-aminoethanol was added dropwise into the four-necked flask using a dropping funnel over 2 hours. After completion of the dropwise addition, stirring was further performed at 70° C. for 3 hours. The composition of the reaction solution was analyzed by GPC according to the above analysis method, revealing that the percentage of the target compound present in the reaction solution was 51 area %.

After completion of the reaction, toluene and water were removed by reduced-pressure distillation at 70° C. The pressure during the distillation was gradually reduced so as to finally reach 4.8 kPa. The composition containing the target compound was taken out, solidified by cooling, then pulverized, and dried at 60° C. and 1.5 kPa to obtain 173 g of the target compound (purity: 53%, compounds with molecular weights higher than that of the target compound: 47 area %).

The results of ¹H-NMR analysis confirmed that a target benzoxazine compound (p-1) having the above structure was obtained.

¹H-NMR analysis (400 MHz, solvent: CDCl₃, reference material: tetramethylsilane)

2.43-2.72 (2H, brm), 2.71-3.16 (4H, m), 3.41-4.09 (12H, m), 4.69-5.01 (4H, m), 6.49-7.07 (6H, m).

### <Example 2> (Synthesis of Compound p-2 by Production Method According to Present Invention)

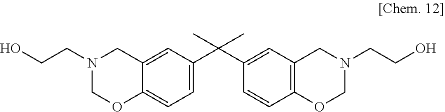 [SMILES: CC(C)(c1ccc2c(c1)CN(CCO)CO2)c1ccc2c(c1)CN(CCO)CO2]

In a 1 L four-necked flask equipped with a thermometer, a stirrer, a condenser, and a dropping funnel, 100 g (0.44 mol) of bisphenol A, 56 g of 94% paraformaldehyde, and 184 g of toluene were loaded. During the loading, heat generation was not observed, and as a result of analyzing the mixed solution by HPLC, only the raw materials used were detected. After the reactor was purged with nitrogen, the temperature of the mixed solution was adjusted to 70° C. While maintaining the temperature, 53 g of 2-aminoethanol was added dropwise into the four-necked flask using a dropping funnel over 2 hours. After completion of the dropwise addition, stirring was further performed at 70° C. for 9.5 hours. The composition of the reaction solution was analyzed by GPC according to the above analysis method, revealing that the percentage of the target compound present in the reaction solution was 52 area %.

After completion of the reaction, toluene and water were removed by reduced-pressure distillation at 70° C. The pressure during the distillation was gradually reduced so as to finally reach 20 kPa. The composition containing the target compound was taken out to obtain 187 g of the target compound (purity: 54%, compounds with molecular weights higher than that of the target compound: 46 area %).

The results of ¹H-NMR analysis confirmed that a target benzoxazine compound (p-2) having the above chemical structure was obtained.

¹H-NMR analysis (400 MHz, solvent: CDCl₃, reference material: tetramethylsilane)

1.14-1.96 (6H, m), 2.45-2.77 (2H, brm), 2.78-3.18 (4H, m), 3.28-4.19 (10H, m), 4.70-5.14 (4H, m), 6.56-7.13 (6H, m).

### <Example 3> (Synthesis 1 of Compound p-3 by Production Method According to Present Invention)

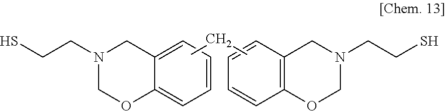

In a 500 mL four-necked flask equipped with a thermometer, a stirrer, a condenser, and a dropping funnel, 31 g (0.15 mol) of bisphenol F, 20 g of 94% paraformaldehyde, and 57 g of toluene were loaded. During the loading, heat generation was not observed, and as a result of analysis by HPLC, only the raw materials used were detected. After the reactor was purged with nitrogen, the temperature of the mixed solution was adjusted to 60° C. While maintaining the temperature, 24 g of 2-aminoethanethiol was added into the four-necked flask over 1 hour. After completion of the addition, stirring was further performed at 60° C. for 2 hours. The composition of the reaction solution was analyzed by GPC according to the above analysis method, revealing that the percentage of the target compound present in the reaction solution was 41 area %.

After completion of the reaction, toluene and water were removed by reduced-pressure distillation at 50° C. The pressure during the distillation was gradually reduced so as to finally reach 2.4 kPa. The composition containing the target compound was taken out to obtain 59 g of the target compound (purity: 41%, compounds with molecular weights higher than that of the target compound: 59 area %).

The results of ¹H-NMR analysis confirmed that a target benzoxazine compound (p-3) having the above chemical structure was obtained.

¹H-NMR (400 MHz, solvent: CDCl₃, reference material: tetramethylsilane)

1.32-1.95 (2H, brm), 2.91-3.05 (4H, m), 3.07-3.22 (4H, m), 3.64-4.13 (10H, m), 6.66-7.12 (6H, m).

### <Example 4> (Synthesis 2 of Compound p-3 by Production Method According to Present Invention)

The reaction was performed in the same manner as in Example 3 except that a 1 L four-necked flask, 97 g (0.48 mol) of bisphenol F, 62 g of 94% paraformaldehyde, 75 g of 2-aminoethanethiol, and 180 g of toluene were used, the temperature before the dropwise addition of amine was adjusted to 50° C., and stirring was further performed at 50° C. for 1 hour after completion of the dropwise addition of amine.

The composition of the reaction solution was analyzed by GPC according to the above analysis method, revealing that the percentage of the target compound present in the reaction solution was 65 area %.

After completion of the reaction, toluene and water were removed by reduced-pressure distillation at 50° C. The pressure during the distillation was gradually reduced so as to finally reach 1.5 kPa. The composition containing the target compound was taken out, solidified by cooling, then pulverized, and vacuum-dried at 60° C. and 1.5 kPa to obtain 208 g of the target compound (purity: 56%, compounds with molecular weights higher than that of the target compound: 44 area %).

### <Example 5> (Synthesis 3 of Compound p-3 by Production Method According to Present Invention)

The reaction was performed in the same manner as in Example 3 except that a 1 L four-necked flask, 97 g (0.48 mol) of bisphenol F, 74 g of 94% paraformaldehyde, 75 g of 2-aminoethanethiol, and 180 g of toluene were used, the temperature before the dropwise addition of amine was adjusted to 30° C., and stirring was further performed at 30° C. for 3 hours after completion of the dropwise addition of amine.

The composition of the reaction solution was analyzed by GPC according to the above analysis method, revealing that the percentage of the target compound present in the reaction solution was 88 area %.

After completion of the reaction, alkali washing was performed using a 3% aqueous sodium hydroxide solution, and water washing was then performed until the pH of the reaction solution became 7 or less. Thereafter, toluene and water were removed by reduced-pressure distillation at 30° C. The pressure during the distillation was gradually reduced so as to finally reach 2.3 kPa. After the solvent was removed to some extent, the remaining solvent was further removed at 90° C. and 2.8 kPa. The composition containing the target compound was taken out, solidified by cooling, and then pulverized to obtain 156 g of the target compound (purity: 75%, compounds with molecular weights higher than that of the target compound: 25 area %).

### <Reference Example> (Confirmation of Formation of Intermediate Having Hexahydrotriazine Structure)

In a 500 mL four-necked flask equipped with a thermometer, a stirrer, and a breather for checking aeration, 36 g of 94% paraformaldehyde, 44 g of 2-aminoethanethiol, and 105 g of toluene were loaded in this order under a stream of nitrogen gas. During the loading, heat generation was observed from the start of the mixing of toluene. The temperature of the mixed solution rapidly increased from 25° C. to 40° C. at about 5 minutes after the mixing, and the inside of the reactor was steamed up. In addition, the rate of bubble generation in the breather slightly increased, which indicated an increase in internal pressure. After stirring for 1 hour, the mixed solution was analyzed by ¹H-NMR, confirming that an intermediate compound having a hexahydrotriazine structure represented by the following structure was formed.

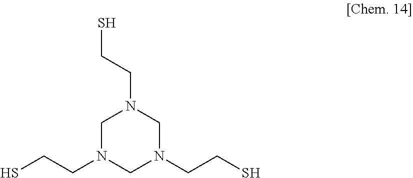 [SMILES: SCCN1CN(CCS)CN(CCS)C1]

¹H-NMR analysis (400 MHz, solvent: CD₃OD, reference material: tetramethylsilane)

2.83 (2H, t), 3.15 (2H, t), 4.17 (2H, s).

From the above, it has been confirmed that an amine compound reacts with a formaldehyde to form an intermediate compound having a hexahydrotriazine structure and that a phenomenon occurs in which the temperature of a mixed solution rapidly increases when raw materials are mixed and smoke generates inside a reactor to cause an increase in internal pressure.

### <Comparative Example 1> (Synthesis of Compound p-3 not by Production Method According to Present Invention)

In a 500 mL four-necked flask equipped with a thermometer, a stirrer, and a condenser, 30 g of 94% paraformaldehyde, 36 g of 2-aminoethanethiol, and 88 g of toluene were loaded. During the loading, as in Reference Example above, the temperature of the mixed solution rapidly increased from 25° C. to 40° C. at about 5 minutes after the mixing, and the inside of the reactor was steamed up.

Then, after the reactor was purged with nitrogen, the mixed solution was heated to 60° C. and mixed with 47 g (0.23 mol) of bisphenol F while maintaining the temperature. After completion of the mixing, stirring was further performed at 60° C. for 3 hours. The composition of the reaction solution was analyzed by GPC according to the above analysis method, revealing that the percentage of the target compound present in the reaction solution was 69 area %.

### <Example 6> (Synthesis of Compound p-5 by Production Method According to Present Invention)

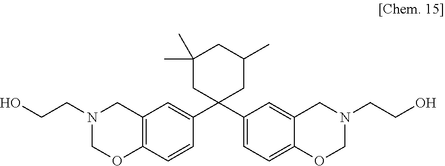 [SMILES: CC1CC(C)(C)CC(c2ccc3c(c2)CN(CCO)CO3)(c2ccc3c(c2)CN(CCO)CO3)C1]

In a 1 L four-necked flask equipped with a thermometer, a stirrer, a condenser, and a dropping funnel, 124 g (0.4 mol) of 1,1-bis(4-hydroxyphenyl)-3,3,5-trimethylcyclohexane, 63 g of 92% paraformaldehyde, and 230 g of toluene were loaded. During the loading, heat generation was not observed, and as a result of analysis by HPLC, only the raw materials used were detected. After the reaction vessel was purged with nitrogen, the temperature of the mixed solution was adjusted to 30° C. While maintaining the temperature, 49 g of 2-aminoethanol was added dropwise into the four-necked flask using a dropping funnel over 2 hours. After completion of the dropwise addition, stirring was further performed at 30° C. for 4 hours. The composition of the reaction solution was analyzed by GPC according to the above analysis method, revealing that the percentage of the target compound present in the reaction solution was 79 area %.

After completion of the reaction, alkali washing was performed using a 3% aqueous sodium hydroxide solution, after which 350 g of toluene was added, and water washing was performed until the pH of the water-washed solution became 7 or less. Thereafter, toluene and water were removed by reduced-pressure distillation at 60° C. The pressure during the distillation was gradually reduced so as to finally reach 4.8 kPa. After the solvent was removed to some extent, the remaining solvent was further removed at 90° C. and 9.8 kPa to obtain 183 g of the target compound (purity: 76%, compounds with molecular weights higher than that of the target compound: 24 area %).

The results of ¹H-NMR analysis confirmed that a target compound (p-5) having the above chemical structure was obtained.

¹H-NMR analysis (400 MHz, solvent: CDCl₃, reference material: tetramethylsilane)

0.30-0.40 (3H, m), 0.84 (1H, m), 0.90-1.00 (6H, m), 1.10 (1H, m), 1.76-2.02 (2H, m), 2.32 (1H, m), 2.58 (1H, m), 2.81-3.07 (4H, m), 3.57-4.05 (8H, m), 4.73-4.90 (4H, m), 6.50-7.12 (6H, m).

### <Example 7> (Synthesis of Compound p-6 by Production Method According to Present Invention)

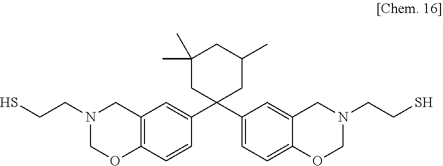 [SMILES: CC1CC(C)(C)CC(c2ccc3c(c2)CN(CCS)CO3)(c2ccc3c(c2)CN(CCS)CO3)C1]

The reaction was performed in the same manner as in Example 3 except that a 1 L four-necked flask equipped with a thermometer, a stirrer, a condenser, and a dropping funnel, 97 g (0.31 mol) of 1,1′-bis(4-hydroxyphenyl)-3,3,5-trimethylcyclohexane, 48 g of 94% paraformaldehyde, 48 g of 2-aminoethanethiol, and 180 g of toluene were used, the temperature before the dropwise addition of amine was adjusted to 30° C., and stirring was further performed at 30° C., 40° C., and 50° C. each for 3 hours after completion of the dropwise addition of amine.

During the mixing of 1,1′-bis(4-hydroxyphenyl)-3,3,5-trimethylcyclohexane and 94% paraformaldehyde, heat generation was not observed, and as a result of analyzing the mixed solution by HPLC, only the raw materials used were detected.

The composition of the reaction solution was analyzed by GPC according to the above analysis method, revealing that the percentage of the target compound present in the reaction solution was 73 area %.

After completion of the reaction, alkali washing was performed using a 3% aqueous sodium hydroxide solution, and water washing was then performed until the pH of the reaction solution became 7 or less. Thereafter, toluene and water were removed by reduced-pressure distillation at 30° C. The pressure during the distillation was gradually reduced so as to finally reach 4.2 kPa. After the solvent was removed to some extent, the remaining solvent was further removed at 90° C. and 20 kPa. The composition containing the target compound was taken out, solidified by cooling, and then pulverized to obtain 188 g of the target compound (purity: 71%, compounds with molecular weights higher than that of the target compound: 29 area %).

The results of ¹H-NMR analysis confirmed that a target compound (p-6) having the above chemical structure was obtained.

¹H-NMR (400 MHz, solvent: CDCl₃, reference material: tetramethylsilane)

0.25-0.44 (3H, m), 0.76-1.02 (7H, m), 1.11 (1H, dd), 1.36 (1H, d), 1.75-2.05 (2H, m), 2.33 (1H, brm), 2.59 (1H, brm), 2.77-3.22 (8H, m), 3.54-3.79 (4H, m), 3.86-4.07 (4H, m), 6.51-7.04 (6H, m), 9.07-10.3 (2H, brm).

1. A method for producing a benzoxazine compound represented by general formula (3), the method comprising performing a reaction by mixing a mixture containing a bisphenol compound represented by general formula (1) and a formaldehyde with an amine compound represented by general formula (2), 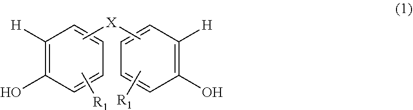
wherein R₁ represents a hydrogen atom or an alkyl group having 1 to 6 carbon atoms, and X represents a single bond, an oxygen atom, a sulfur atom, a sulfonyl group, a carbonyl group, or a divalent group represented by general formula (1a) or (1b), 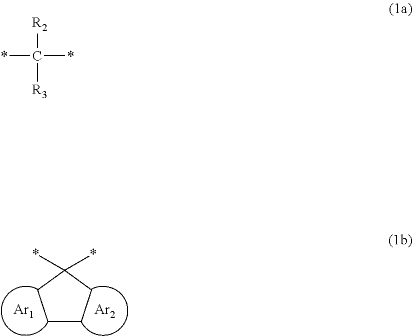
wherein, in general formulae (1a) and (1b), R₂ and R₃ each independently represent hydrogen, an alkyl group having 1 to 10 carbon atoms, an alkyl halide group having 1 to 10 carbon atoms, or an aryl group having 6 to 12 carbon atoms, R₂ and R3 are optionally bonded to each other to together form a cycloalkylidene group having 5 to 20 carbon atoms, Ar₁ and Ar₂ each independently represent an aryl group having 6 to 12 carbon atoms, and * represents a bonding position,
H₂N—R₄—Y (2)
wherein R₄ represents a divalent group having 1 to 10 carbon atoms, and Y represents a hydroxy group or a thiol group, 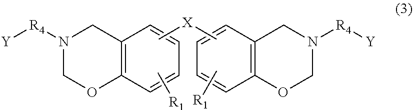
wherein R₁ and X represent the same groups as in general formula (1), and R₄ and Y represent the same groups as in general formula (2).
